(19) United States
(12) Patent Application Publication
(10) Pub. No.: US 2013/0079655 A1
(43) Pub. Date: Mar. 28, 2013

(54) PORTABLE ELECTROCARDIOGRAPH

(71) Applicant: Wei HSU, New Taipei City (TW)

(72) Inventor: Wei HSU, New Taipei City (TW)

(21) Appl. No.: 13/342,115

(22) Filed: Jan. 2, 2012

(30) Foreign Application Priority Data
Sep. 22, 2011 (TW) 100217796

(51) Int. Cl. A61B 5/0432; A61B 5/0408; A61B 5/044

(52) U.S. Cl. 600/523

(57) ABSTRACT

A portable electrocardiograph includes a sensing unit and an process platform. The sensing unit having a signal wire is arranged to a patient for sensing the cycle of heart beat. The process platform having an application program serves to process and analyze the cycle of heart beat sensed by the sensing unit, the process result wil' be recorded and displayed. The process result will be transmitted through wire, wireless, Bluetooth transmission so that the initial symptom can be delivered to medical personnel. The process result can also be transmitted to data base of hospital server through network for advanced analysis so as to perform further diagnostic and treatment. The portable electrocardiograph is easily carried by patients so as to apply a self-diagnostic in time and inform medical personnel the latest condition for a complete diagnostic.

### FIELD OF THE INVENTION

The present invention relates to distanced medical care, and particular to a portable electrocardiograph.

### DESCRIPTION OF THE PRIOR ART

Prior treatments for patients suffer heart disease, pant, blood disease are periodic diagnostic, self-measurement for tracking the condition of the patients. The data of the self-measurement must be analyzed by instruments such as an electrocardiograph for diagnostic. However, prior electrocardiograph in a hospital is large and needs to be operated by medical worker. Time and latest condition of the patient can not be saved and delivered to medical personnel. Thus, long-distance medical care services such as simple electrocardiogram recorder are provided for tracking the condition of the patient. The simple portable device still needs to improve. With the convenient and advanced information transmission nowadays, those portable devices combined to a computer or cell phone can deliver real-time electrocardiogram and the analysis result to medical center and the patient himself for alert and proper medical treatment.

### SUMMARY OF THE PRESENT INVENTION

Accordingly, the primary object of the present invention is to provide a portable electrocardiograph to alert patient of the condition for reference of medical treatment. The most complete information of the patient can be delivered to medical personnel in time to save time. The initial condition of the patient can be also preserved by self-diagnostic of the present invention.

To achieve above objects, the present invention includes the following components.

A sensing unit can be arranged to a patient for measure cycle of heart beat. The sensing unit has a signal wire.

A process platform has an application program to analyze the heart beat cycle measure by the sensing unit. The analysis result can be recorded and shown.

The process result can be transmitted through wire, wireless, Bluetooth transmission so that the initial condition can be delivered to medical personnel. The process result can also be transmitted to data base of hospital server through network for advanced analysis so as to perform further diagnostic and treatment.

The portable electrocardiograph is easily carried by patients so as to apply a self-diagnostic in time and inform medical personnel the latest condition for a complete diagnostic.

The various objects and advantages of the present invention will be more readily understood from the following detailed description when read in conjunction with the appended drawing.

### BRIEF DESCRIPTION OF THE DRAWINGS

FIG. 1 is a system diagram of a portable electrocardiograph of the present invention.

FIG. 2 is a system diagram of a sensing unit of the portable electrocardiograph of the present invention.

FIG. 3 is a system diagram of a process platform of the portable electrocardiograph of the present invention.

FIG. 4 is a flow chart showing the operation of the portable electrocardiograph of the present invention.

FIG. 5 is a system diagram of a medical diagnostic platform referred in FIG. 4.

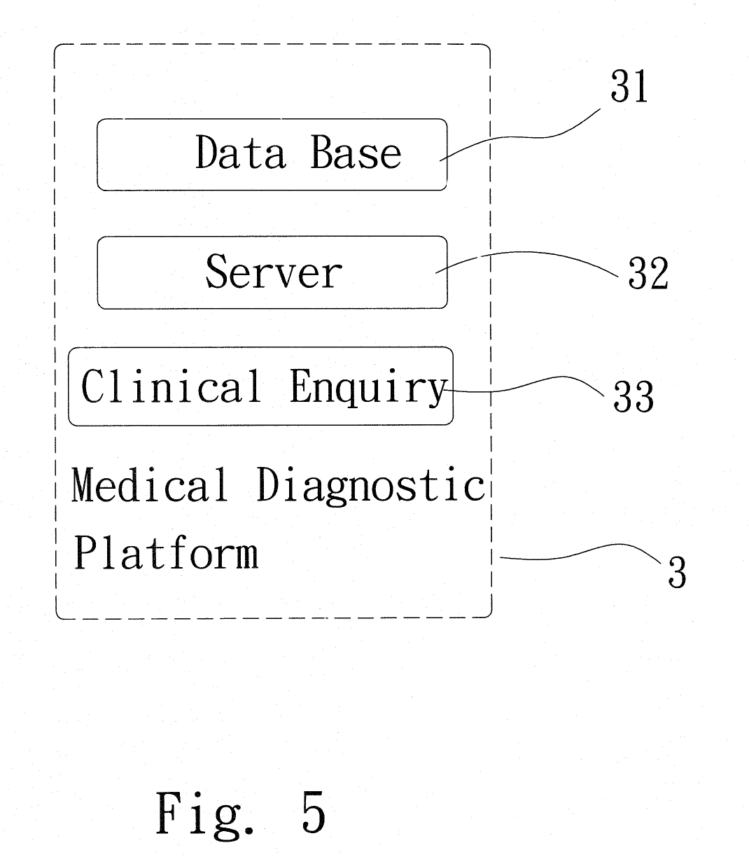

### DETAILED DESCRIPTION OF THE IVENTION

In order that those skilled in the art can further understand the present invention, a description will be provided in the following in details. However, these descriptions and the appended drawings are only used to cause those skilled in the art to understand the objects, features, and characteristics of the present invention, but not to be used to confine the scope and spirit of the present invention defined in the appended claims.

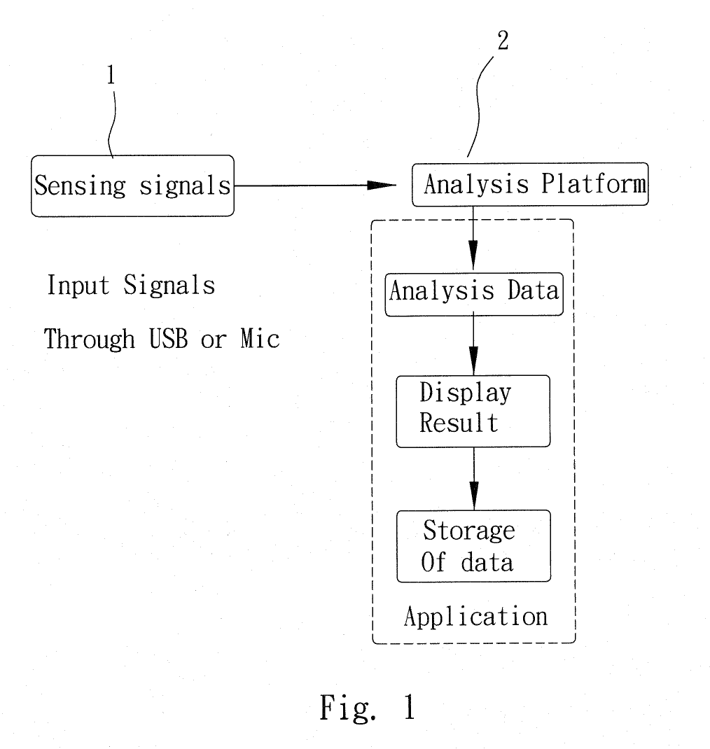

Referring to FIG. 1, a preferable embodiment of a portable electrocardiograph according to the present invention includes following main components.

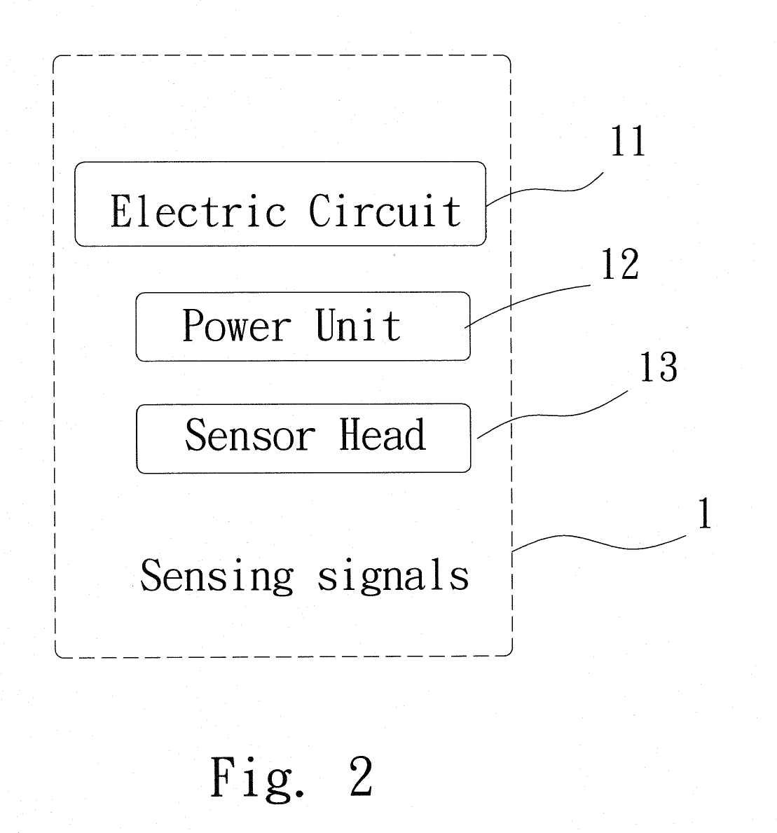

A sensing unit 1 is arranged to a patient by specific means to measure cycle of heart beat as shown in FIG. 2. The sensing unit 1 further includes an electric circuit 11, power unit 12, and a sensor head 13. The heart beat sensed by the sensor head 13 will be transmitted to the electric circuit 11 for analysis and record. The sensing signal 1 has a signal wire, the signal wire uses USB, MIC, or any other connection interface.

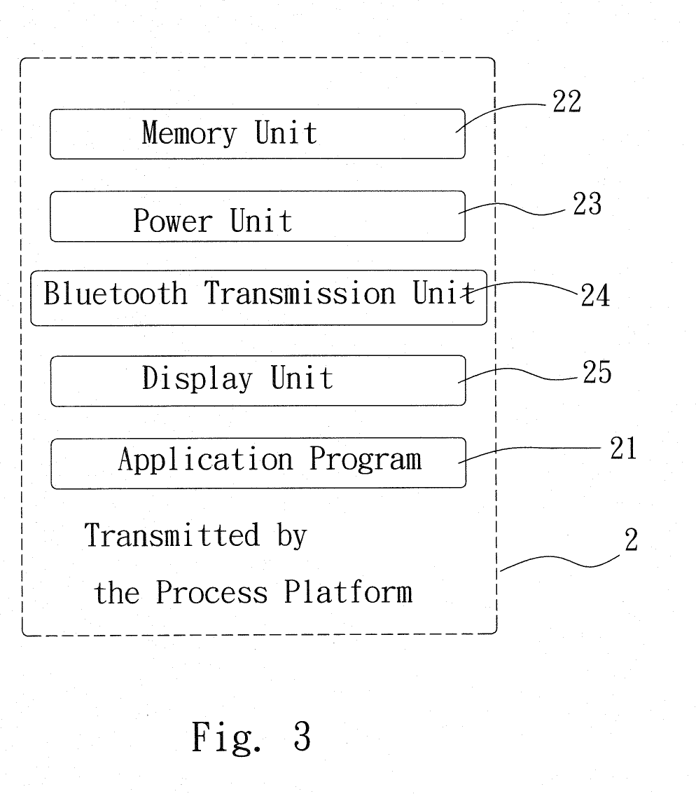

A process platform 2 having a application program 21 serves to process and analyze the signal sensed by the sensing unit 1, the process result will be stored and displayed as shown in FIG. 3. The process platform 2 further includes a memory unit 22, power unit 23, network/Bluetooth transmission unit 24, and a display unit 25. The memory unit 22 serves to store the signal sensed by the sensing unit 1. The power unit 23 serves to supply power. The network/Bluetooth transmission unit 24 serves to transmit the process result of the process platform 2 to hospital, consulter, or a cloud server. The display unit 25 serves to show the process result of the process platform 2 to the patient.

Moreover, the process platform 2 can be a computer of Windows, Linux, Android, or Mac system, smart phone, specific hardware, or any other equipment.

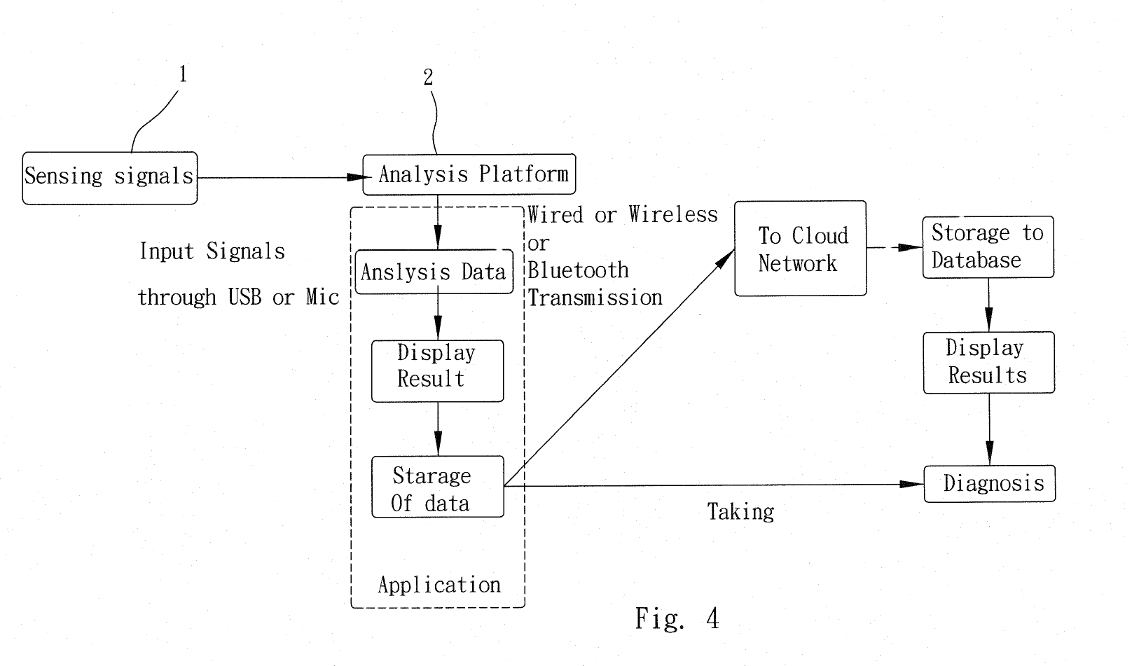

Referring to FIG. 4, the sensing unit 1 is arranged to a patient. When the patient is ill, the sensing unit 1 can be connected to the process platform 2 through the signal wire. The application program 21 of the process platform 2 can process and analyze the signal, the process result will be stored in the memory unit 22 and displayed on the display unit 25 so that the initial symptom can be delivered to medical personnel. The process result can be transmitted by the network/Bluetooth transmission unit 24 to hospital, consulter, or cloud server through wire or wireless transmission. The process result can be recorded for advanced analysis so as to perform further diagnostic and treatment.

A medical diagnostic platform 3 includes a data base 31, server 32, and clinical enquiry 33. The data base 31 serves to store the process result transmitted by the process platform 2. The server 32 can be accessed by patients for their analysis results. Patients can also have complete diagnostic through direct clinical enquiry 33. The medical diagnostic platform 3 can also be a computer of Windows, Linux, Android, or Mac system, smart phone, specific hardware, or any other equipment.

Through the above components, the present invention can achieve the following achievements.

1. The portable electrocardiograph is easily carried by patients to apply a self-diagnostic in time and inform medical personnel the latest condition for diagnostic.
2. A portable process platform of computer or cell phone capable of analyzing and showing the result of electrocardiogram can record the initial condition and symmetrically transmit the data to medical center so as to alert the patient of the condition and shorten the diagnostic for medical personnel.
3. Latest condition of patient can be deled to medical server through wire, wireless, Bluetooth transmission so that the condition of patient can be tracked and the enquiry efficiency can be raised.

The present invention is thus described, it will be obvious that the same may be varied in many ways. Such variations are not to be regarded as a departure from the spirit and scope of the present invention, and all such modifications as would be obvious to one skilled in the art are intended to be included within the scope of the following claims.

What is claimed is:

1. A portable electrocardiograph comprising:
a sensing unit arranged to a patient for measure cycle of heart beat; the sensing unit having a signal wire;
a process platform having an application program to analyze the heart beat cycle; the analysis result being recorded and shown by the process platform.

2. The portable electrocardiograph as claimed in claim 1, wherein the sensing unit is arranged to patient by any means to measure heart beat cycle.

3. The portable electrocardiograph as claimed in claim 1, wherein the sensing unit includes a electric circuit, power unit, and a sensor head.

4. The portable electrocardiograph as claimed in claim 1, wherein the interface of the signal wire is one of USB or MIC.

5. The portable electrocardiograph as claimed in claim 1, wherein the process platform includes a memory unit, power unit, network/Bluetooth transmission unit, and a display unit.

6. The portable electrocardiograph as claimed in claim 1, wherein the network/Bluetooth transmission unit transmits the analysis result to a medical server through one of wire, wireless, or Bluetooth transmission.

7. The portable electrocardiograph as claimed in claim 6, wherein the medical server includes a medical diagnostic platform having a data base, server, and clinical enquiry service.

8. The portable electrocardiograph as claimed in claim 7, wherein the medical diagnostic platform is one of a computer of Windows, Linux, Android, or Mac system, or a specific hardware.

9. The portable electrocardiograph as claimed in claim 1, wherein the process platform is one of a computer of Windows, Linux, Android, or Mac system, smart phone, specific hardware.
